(19) United States
(12) Patent Application Publication
(10) Pub. No.: US 2007/0202060 A1
(43) Pub. Date: Aug. 30, 2007

(54) Retinoid-Containing Preparations

(75) Inventors: Arne Ptock, Ludwigshafen (DE); Axel Jentzsch, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft Patents, Trademarks and Licenses, Ludwigshafen (DE)

(21) Appl. No.: 10/586,776

(22) PCT Filed: Jan. 14, 2005
(86) PCT No.: PCT/EP05/00312

§ 371 Date: Jul. 21, 2006

(30) Foreign Application Priority Data
Jan. 22, 2004 (DE) 10 2004 003 478.8

(51) Int. Cl. A61K 8/49; A61K 31/355; A61K 31/07

(52) U.S. Cl. 424/59; 514/474; 514/458; 514/725

(57) ABSTRACT

Preparations comprising a. at least one retinoid, b. at least one water-soluble antioxidant, c. at least one oil-soluble antioxidant and d. 0.01 to 10% by weight of at least one UV filter, wherein, in the preparations, per part by weight of retinoid, at least 1 part by weight of one or more water-soluble antioxidants and 0.1 to 100 parts by weight of one or more oil-soluble antioxidants are present, where the content of one or more water-soluble antioxidants is in the range from 0.05 to 0.8% by weight, based on the total amount of the preparations.

The present invention relates to retinoid-containing preparations, to their preparation and use in cosmetics and pharmacy.

Retinoids are some of the most active ingredients which are used in cosmetics and in dermatology. They have, inter alia, a regulating effect on normal cell growth and influence the differentiation of epithelial cells. Retinoic acids are thus used for the treatment of acne and retinol is used in antiwrinkle creams.

However, the use of retinoids is severely restricted, which can be attributed, inter alia, to the high instability of the compounds. For this reason, strict safety precautions have to be observed during the production of retinoid-containing preparations. For example, the production must take place entirely under protective gas and the finished product must be packaged in light- and oxygen-impermeable packaging.

These requirements demand that the manufacturer has complex technological equipment and are thus associated with high production costs.

A number of methods of stabilizing retinoids have been described. For example EP-A-1 055 720 discloses the stabilization of oxygen-sensitive compounds by using thio compounds or glycoproteins with the exclusion of oxygen.

According to WO 93/00085 and EP-A-0 440 398, both water- and also fat-soluble antioxidants are used together with chelate-forming agents for the stabilization of retinoids.

However, the methods described do not always lead to adequate stabilization for the purposes of the invention. Instead, both water-soluble antioxidants, in particular ascorbic acid, and fat-soluble antioxidants, in particular tocopherol, exhibit a destabilizing effect in connection with retinoids in certain concentration ranges.

In addition, under some circumstances, the use of certain antioxidant combinations together with retinoids may lead to undesired secondary effects, e.g. yellowish discolorations of the preparations, which render the use of these systems in cosmetics or in the food sector unfeasible.

It was therefore an object of the present invention to provide retinoid-containing preparations which do not have the disadvantages given above with regard to stability and discoloration, and which can be produced in a simple manner.

This object was achieved by preparations comprising

a. at least one retinoid,

b. at least one water-soluble antioxidant,

c. at least one oil-soluble antioxidant and

d. 0.01 to 10% by weight of at least one UV filter,

wherein, in the preparations, per part by weight of retinoid, at least 1 part by weight of one or more water-soluble antioxidants and 0.1 to 100 parts by weight of one or more oil-soluble antioxidants are present, where the content of one or more water-soluble antioxidants is in the range from 0.05 to 0.8% by weight, based on the total amount of the preparations.

The preparations according to the invention are cosmetic and dermatological or pharmaceutical preparations. Preference is given to cosmetic preparations, in particular skincare preparations.

An advantageous embodiment of the preparations according to the invention comprises, per part by weight of retinoid, 1 to 100 parts by weight, preferably 1 to 20 parts by weight, particularly preferably 1 to 6 parts by weight, very particularly preferably 3 to 5 parts by weight, of one or more water-soluble antioxidants and 1 to 20 parts by weight, preferably 1 to 15 parts by weight, particularly preferably 1 to 10 parts by weight, very particularly preferably 3 to 5 parts by weight, of one or more oil-soluble antioxidants.

For the purposes of the present invention, retinoids means vitamin A alcohol (retinol) and its derivatives, such as vitamin A aldehyde (retinal), vitamin A acid (retinoic acid) and vitamin A esters, such as retinyl acetate and retinyl palmitate. The term retinoic acid here comprises both all-trans retinoic acid and 13-cis retinoic acid. The terms retinol and retinal preferably comprise the all-trans compounds. The preferred retinoid used for the preparations according to the invention is all-trans-retinol.

The water-soluble antioxidants intended are, inter alia, ascorbic acid, sodium sulfite, sodium metabisulfite, sodium bisulfite, sodium thiosulfite, sodium formaldehyde sulfoxide, isoascorbic acid, thioglycerol, thiosorbitol, thiourea, thioglycolic acid, cysteine hydrochloride, 1,4-diazobicyclo(2,2,2)octane or mixtures thereof.

Preferred water-soluble antioxidants are ascorbic acid (L-ascorbic acid) and isoascorbic acid (D-ascorbic acid), particularly preferably L-ascorbic acid.

The L-ascorbic acid which is particularly preferably used may be the free acid or else salts thereof. Examples of salts of L-ascorbic acid are alkali metal or alkaline earth metal salts of L-ascorbic acid, such as sodium L-ascorbate, potassium L-ascorbate or calcium L-ascorbate, but also salts of L-ascorbic acid with an organic amine compound, such as choline ascorbate or L-carnitine ascorbate. Very particular preference is given to using the free L-ascorbic acid or sodium L-ascorbate. Corresponding statements apply to the use of D-ascorbic acid.

The oil-soluble antioxidants intended are, inter alia, butylated hydroxytoluene (BHT), ascorbyl palmitate, butylated hydroxyanisol, α-tocopherol, phenyl-α-naphthylamine or mixtures thereof.

A preferred oil-soluble antioxidant is α-tocopherol, which may either be (R,R,R)- or (all-rac)-α-tocopherol.

For the purposes of the present invention, UV filter means UV-A, UV-B and/or broadband filters.

Advantageous broadband filters, UV-A or UV-B filter substances are, for example, representatives of the following compound classes:
bis-resorcinyltriazine derivates with the following structure: 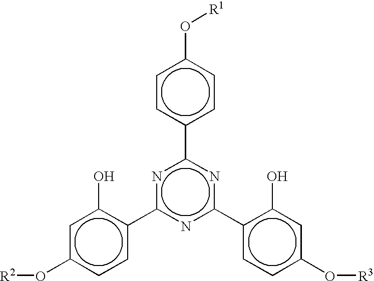
where R¹, R² and R³, independently of one another, are chosen from the group of branched and unbranched alkyl groups having 1 to 10 carbon atoms or an individual hydrogen atom. Particular preference is given to 2,4-bis-{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH.

Other UV filter substances, which have the structural motif 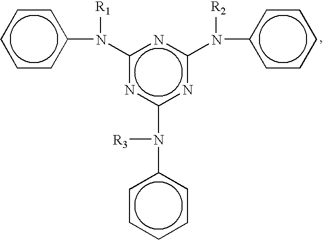
are also advantageous UV filter substances within the meaning of the present invention, for example the s-triazine derivatives described in the European laid-open specification EP 570 838 A1, the chemical structure of which is given by the generic formula 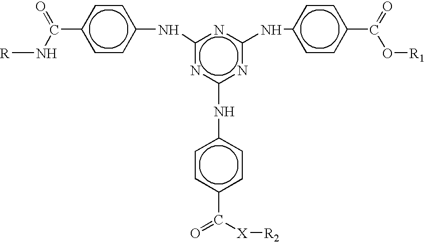
where
R is a branched or unbranched C₁-C₁₈-alkyl radical, a C₅-C₁₂-cycloalkyl-radical, optionally substituted by one or more C₁-C₄-alkyl groups,
X is an oxygen atom or an NH group,
R₁ is a branched or unbranched C₁-C₁₈-alkyl radical, a C₁-C₁₂-cycloalkyl radical, optionally substituted by one or more C₁-C₄-alkyl groups, or a hydrogen atom, an alkali metal atom, an ammonium group or a group of the formula 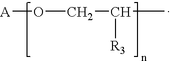
   in which
A is a branched or unbranched C₁-C₁₈-alkyl radical, a C₁-C₁₂-cycloalkyl or aryl radical, optionally substituted by one or more C₁-C₄-alkyl groups,
R₃ is a hydrogen atom or a methyl group,
n is a number from 1 to 10,
R₂ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₁-C₁₂-cycloalkyl radical, optionally substituted by one or more C₁-C₄-alkyl groups, or a hydrogen atom, an alkali metal atom, an ammonium group or a group of the formula 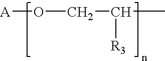
   in which
A is a branched or unbranched C₁-C₁₈-alkyl radical, a C₁-C₁₂-cycloalkyl or aryl radical, optionally substituted by one or more C₁-C₄-alkyl groups,
R₃ is a hydrogen atom or a methyl group,
n is a number from 1 to 10,
   if X is an oxygen atom.

A particularly preferred UV filter substance within the meaning of the present invention is also an asymmetrically substituted s-triazine whose chemical structure is given by the formula 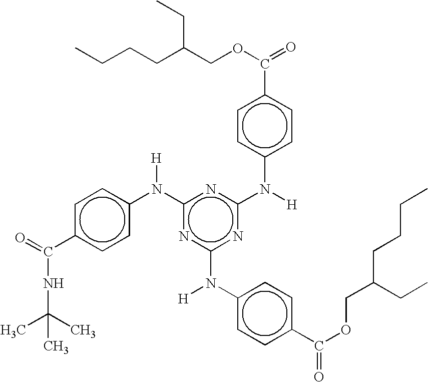
which is also referred to below as dioctylbutylamidotriazone (INCI: Diethylhexylbutamidotriazone) and is available under the trade name UVASORB® HEB from Sigma 3V.

Also advantageous within the meaning of the present invention is a symmetrically substituted s-triazine, tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® 150.

European laid-open specification 775 698 also describes bisresorcinyltriazine derivates to be used with preference, the chemical structure of which is given by the generic formula 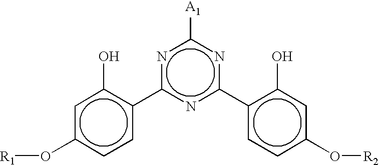
where R₁ and R₂ are, inter alia, C₃-C₁₈-alkyl or C₂-C₁₈-alkenyl and A₁ is an aromatic radical.

Also advantageous for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethyl-siloxysilylpropyloxy)-2-hydroxy]phenyl}-6-(4-methoxyphenyl)-1, 3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

Advantageous sulfonated water-soluble UV filters for the purposes of the present invention are:
phenylene-1,4-bis(2-benzimidazyl)-3,3′-5,5′-tetrasulfonic acid, which is characterized by the following structure: 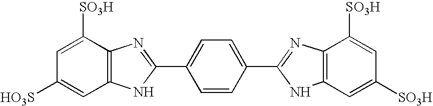
and its salts, particularly the corresponding sodium, potassium or triethanolammonium salts, in particular phenylene-1,4-bis(2-benzimidazyl)-3,3′-5,5′-tetrasulfonic acid bis-sodium salt 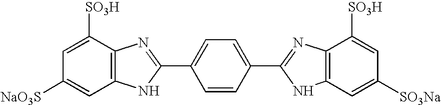
with the INCI name Bisimidazylate (CAS No.: 180898-37-7), which is obtainable, for example, under the trade name Neo Heliopan® AP from Haarmann & Reimer.

A further sulfonated UV filter advantageous for the purposes of the present invention are the salts of 2-phenylbenzimidazole-5-sulfonic acid, such as its sodium, potassium or its triethanolammonium salt, and the sulfonic acid itself 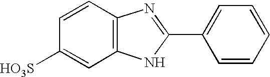
with the INCI name Phenylbenzimidazole Sulfonic Acid (CAS No. 27503-81-7), which is obtainable, for example, under the trade name Eusolex® 232 from Merck or under Neo Heliopan® Hydro from Haarmann & Reimer.

A further advantageous sulfonated UV filter is 3,3′-(1,4-phenylenedimethylene)bis(7,7-dimethyl-2-oxobicyclo[2.2.1]hept-1-ylmethanesulfonic acid, such as its sodium, potassium or triethanolammonium salt, and the sulfonic acid itself: 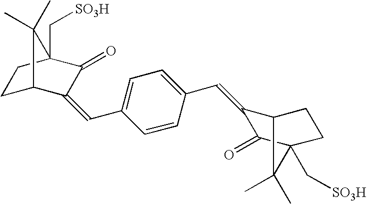
with the INCI name Terephtalidene Dicamphor Sulfonic Acid (CAS No.: 90457-82-2), which is obtainable, for example, under the trade name Mexoryl® SX from Chimex.

Further advantageous water-soluble UV-B and/or broadband filter substances are e.g.:

sulfonic acid derivatives of 3-benzylidenecamphor, such as e.g. 4-(2-oxo-3-bornylidenemethyl)benzenesulfonic acid, 2-methyl-5-(2-oxo-3-bornylidenemethyl)sulfonic acid and salts thereof.

The UV-B and/or broadband filters may be oil-soluble or water-soluble. Advantageous oil-soluble UV-B and/or broadband filter substances are e.g.:

3-benzylidenecamphor derivatives, preferably 3-(4-methylbenzylidene)camphor, 3-benzylidenecamphor;

4-aminobenzoic acid derivatives, preferably (2-ethylhexyl) 4-(dimethylamino)benzoate, amyl 4-(dimethylamino)benzoate, polyethyoxyethyl 4-bis(polyethoxy)aminobenzoate (obtainable under the trade name Uvinul® P25 from BASF);

Derivatives of benzophenone, preferably 2-hydroxy-4-methoxybenzophenone (obtainable under the trade name Uvinul® M40 from BASF), 2-hydroxy-4-methoxy-4′-methylbenzophenone, 2,2′-dihydroxy-4-methoxybenzophenone, 2-hydroxy-4-methoxybenzophenone-5-sulfonic acid (obtainable under the trade name Uvinul® MS40 from BASF), 2,2′,4,4′-tetrahydroxybenzophenone (obtainable under the trade name Uvinul® D 50 from BASF);

and UV filters bonded to polymers.

For the purposes of the present invention, particularly advantageous UV filter substances which are liquid at room temperature are homomethyl salicylate, 2-ethylhexyl 2-cyano-3,3-diphenylacrylate, 2-ethylhexyl 2-hydroxybenzoate and esters of cinnamic acid, preferably 2-ethylhexyl 4-methoxycinnamate and isopentyl 4-methoxycinnamate.

Homomethyl salicylate (INCI: Homosalate) is characterized by the following structure: 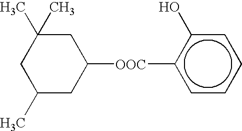
2-ethylhexyl 2-cyano-3,3-diphenylacrylate (INCI: Octocrylene) is obtainable from BASF under the name Uvinul® N 539T and is characterized by the following structure:

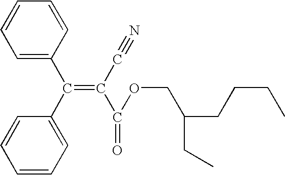

2-ethylhexyl 2-hydroxybenzoate (2-ethylhexyl salicylate, octyl salicylate, INCI: Ethylhexyl Salicylate) is obtainable, for example, from Haarmann & Reimer under the trade name Neo Heliopan® OS and is characterized by the following structure:

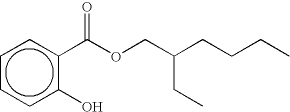

2-ethylhexyl 4-methoxycinnamate (INCI: Ethylhexyl Methoxycinnamate) is obtainable, for example, from BASF under the trade name Uvinul® MC 80 and is characterized by the following structure:

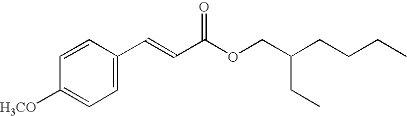

Isopentyl 4-methoxycinnamate (INCI: Isoamyl p-Methoxycinnamate) is obtainable, for example, from Haarmann & Reimer under the trade name Neo Heliopan® E 1000 and is characterized by the following structure:

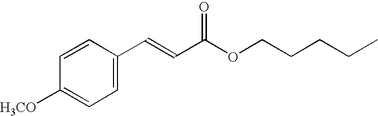

For the purposes of the present invention, a further advantageous UV filter substance which is liquid at room temperature (3-(4-(2,2-bisethoxycarbonylvinyl)phenoxy)propenyl)-methylsiloxane/dimethylsiloxane copolymer, which is obtainable, for example, from Hoffmann-La Roche under the trade name Parsol® SLX.

For the purposes of the present invention, advantageous dibenzoylmethane derivatives are in particular 4-(tert-butyl)-4′-methoxydibenzoylmethane (CAS No. 70356-09-1), which is sold by BASF under the brand Uvinul® BMBM and by Merck under the trade name Eusolex® 9020, which is characterized by the following structure:

A further advantageous 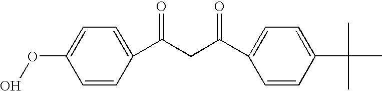
advantageous dibenzoylmethane derivative is 4-isopropyldibenzoylmethane (CAS No. 63250-25-9), which is sold by Merck under the name Eusolex® 8020. Eusolex 8020 is characterized by the following structure:

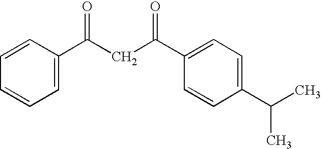

Benzotriazoles are characterized by the following structural formula: 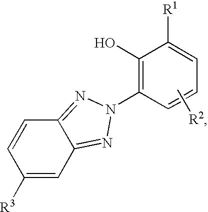
in which
R¹ and R², independently of one another, can be linear or branched, saturated or unsaturated, substituted (e.g. substituted by a phenyl radical) or unsubstituted alkyl radicals having 1 to 18 carbon atoms and/or polymer radicals which themselves do not absorb UV rays (such as e.g. silicon radicals, acrylate radicals and the like), and R₃ is chosen from the group H or alkyl radical having 1 to 18 carbon atoms.

For the purposes of the present invention, an advantageous benzotriazole is 2,2′-methylenebis(6-(2H-benzotriazol-2-yl)-4-(1,1,3,3-tetramethylbutyl)phenol), a broadband filter which is characterized by the chemical structural formula 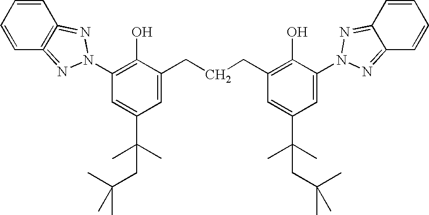
and is obtainable under the trade name Tinosorb® M from CIBA-Chemikalien GmbH.

For the purposes of the present invention, another advantageous benzotriazole is 2-(2H-benzotriazol-2-yl)-4-methyl-6-[2-methyl-3-[1,3,3,3-tetramethyl-1-[(trimethylsilyl)oxy]disiloxanyl]propyl]-phenol (CAS No.: 155633-54-8) with the INCI name Drometrizole Trisiloxane, which is sold by Chimex under the brand Mexoryl® XL and is characterized by the following chemical structural formula

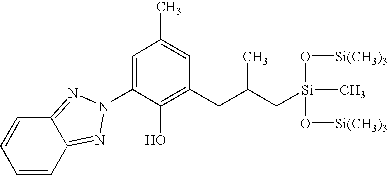

For the purposes of the present invention, further advantageous benzotriazoles are [2,4′-dihydroxy-3-(2H-benzotriazol-2-yl)-5-(1,1,3,3-tetramethylbutyl)-2′-n-octoxy-5′-benzoyl]diphenyl-methane, 2,2′-methylenebis[6-(2H-benzotriazol-2-yl)-4-(methyl)phenol], 2,2′-methylenebis-[6-(2H-benzotriazol-2-yl)-4-(1,1,3,3-tetramethylbutyl)phenol], 2-(2′-hydroxy-5′-octyl-phenyl)benzotriazole, 2-(2′-hydroxy-3′,5′-di-t-amylphenyl)benzo-triazole and 2-(2′-hydroxy-5′-methylphenyl)benzotriazole.

For the purposes of the present invention, a further advantageous UV filter is the diphenylbutadiene compound of the following formula described in EP-A-O 916 335.

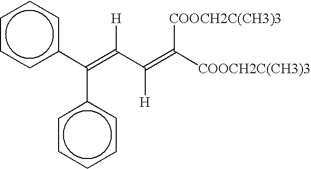

For the purposes of the present invention, a further advantageous UV-A filter is the diethyl 2-(4-ethoxyanilinomethylene)propanedicarboxylate of the following formula described in EP-A-O 895 776

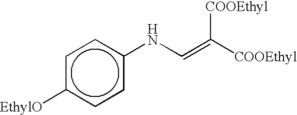

Likewise advantageous for the purposes of the present invention is an amino-substituted hydroxybenzophenone of the following formula: 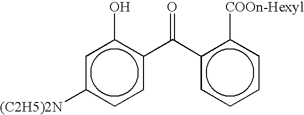
which is sold by BASF Aktiengesellschaft as UV-A filter under the trade name UVINUL® A Plus.

Cosmetic and dermatological preparations according to the invention also advantageously, although not necessarily, comprise inorganic pigments based on metal oxides and/or other metal compounds which are sparingly soluble or insoluble in water, in particular the oxides of titanium (TiO₂), zinc (ZnO), iron (e.g. Fe2O3), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. These pigments are X-ray amorphous or non-X-ray amorphous. They are particularly preferably pigments based on TiO2.

X-ray amorphous oxide pigments are metal oxides or semimetal oxides which, in X-ray diffraction experiments, reveal no or no recognizable crystal structure. Such pigments are often obtainable by flame reaction, for example by reacting a metal halide or semimetal halide with hydrogen and air (or pure oxygen) in a flame.

In cosmetic, dermatological or pharmaceutical formulations, X-ray amorphous oxide pigments are used as thickeners and thixotropic agents, flow auxiliaries, for emulsion and dispersion stabilization and as carrier substance (for example for increase in the volume of finely divided powders).

X-ray amorphous oxide pigments which are known and often used in cosmetic or dermatological galenics are the silicon oxides of the Aerosil® type (CAS No. 7631-86-9. Aerosils®, obtainable from DEGUSSA, are characterized by small particle size (e.g. between 5 and 40 nm), where the particles are to be regarded as being spherical particles of very uniform dimension. Macroscopically, Aerosils® are recognizable as loose, white powders. For the purposes of the present invention, X-ray amorphous silicon dioxide pigments are particularly advantageous, and among these in particular those of the Aerosil® type are preferred.

Advantageous Aerosil® grades are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974, Aerosil® R976.

According to the invention, the non-X-ray amorphous inorganic pigments are advantageously in hydrophobic form, i.e. have been superficially treated to repel water. This surface treatment can consist in providing the pigments with a thin hydrophobic layer by methods known per se.

Such a method consists, for example, in producing the hydrophobic surface layer by a reaction according to
nTiO₂+m(RO)3Si—R′→nTiO₂(surf.)
n and m here are stoichiometric parameters to be used as desired, R and R′ are the desired organic radicals. Hydrophobized pigments synthesized, for example, in analogy to DE-A 33 14 742 are advantageous.

For the purposes of the present invention, organic surface coatings can consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel), octyltrimethoxysilane or alginic acid. These organic surface coatings can occur on their own, in combination and/or in combination with inorganic coating materials.

Zinc oxide particles and predispersions of zinc oxide particles suitable according to the invention are obtainable under the following trade names from the companies listed:

| Trade name | Coating | Manufacturer |
|---|---|---|
| Z-Cote ® HP1 | 2% Dimethicone | BASF |
| Z-Cote ® | — | BASF |
| ZnO NDM | 5% Dimethicone | H&R |
| MZ-505 S | 5% Methicone | Tayca Corp. |

Suitable titanium dioxide particles and predispersions of titanium dioxide particles are obtainable under the following trade names from the companies listed:

| Trade name | Coating | Manufacturer |
|---|---|---|
| MT-100TV | Aluminum hydroxide/stearic | Tayca Corporation |
| | acid | |
| MT-100Z | Aluminum hydroxide/stearic | Tayca Corporation |
| | acid | |
| Eusolex ® T-2000 | Alumina/simethicone | Merck KgaA |
| Titanium dioxide | Octyltrimethoxysilane | Degussa, BASF |
| T805 (Uvinul ® | | |
| TiO₂) | | |

Advantageous TiO₂ pigments are obtainable, for example, under the trade name Uvinul® TiO₂, advantageous TiO₂/Fe₂O₃ mixed oxides under the trade name Uvinul® TiO₂ A from BASF.

The total amount of at least one UV filter used in the finished cosmetic or dermatological preparations is advantageously chosen from the range from 0.01 to 10% by weight, preferably in the range from 0.5 to 8% by weight, particularly preferably in the range from 1 to 7% by weight, based on the total weight of the preparations.

The UV filters present in the preparations according to the invention can be used here both for stabilizing retinol (product protection) and for protecting the human skin against UV radiation (skin protection).

For the use as product protection, the required amount of UV filters to be used is reduced significantly and for these cases is advantageously in the range from 0.01 to 0.5% by weight, preferably in the range from 0.05 to 0.1% by weight, based on the total weight of the preparations.

The UV filters to be named as preferred for the use in the preparations according to the invention are the following Uvinul® brands from BASF: Uvinul® A Plus, Uvinul® D 50, Uvinul® M 40, Uvinul® MS 40 and Uvinul® P 25, Uvinul® MC 80, Uvinul® N 539, Uvinul® T150 and the inorganic pigments TiO₂ and ZnO.

Particularly for use as product protection, the following UV filters may be specified: Uvinul® A Plus, Uvinul® D 50, Uvinul® M 40, Uvinul® MS 40 and Uvinul® P 25.

The preparations according to the invention usually comprise 0.015 to 0.2% by weight, preferably 0.02 to 0.15% by weight, particularly preferably 0.03 to 0.15% by weight, very particularly preferably 0.04 to 0.12% by weight, of one or more retinoids, in particular all-trans-retinol, 0.05 to 0.8% by weight, preferably 0.08 to 0.7% by weight, particularly preferably 0.12 to 0.6% by weight, very particularly preferably 0.16 to 0.5% by weight, of one or more water-soluble antioxidants, in particular L-ascorbic acid and 0.0005 to 2% by weight, preferably 0.01 to 1.8% by weight, particularly preferably 0.1 to 1.5% by weight, very particularly preferably 0.15 to 1.2% by weight, of one or more oil-soluble antioxidants, in particular α-tocopherol.

The preparations according to the invention are characterized inter alia in that it is possible to dispense with the use of protective gas during their production, bottling and storage while simultaneously ensuring adequate stability.

For the purposes of the present invention, adequate stability is understood as meaning that the retinoid is found again in an amount of at least 90% in the preparation after storage for 12 weeks at 40° C. In addition, no undesired color changes arise during storage of the preparations according to the invention.

It is advantageous if the preparations according to the invention are stored in oxygen-impermeable packagings.

The oxygen-impermeable packagings may be any standard commercial packagings suitable for this purpose, such as e.g. glass containers or aluminum packagings.

A further advantage of the preparations according to the invention is that these products no longer have to be stored under the exclusion of light.

By comparison with the hitherto known retinoid-containing cosmetic preparations, when using the preparations according to the invention in skincare it is now possible to correct the increased sensitivity of the skin to sunlight often triggered by the retinoid.

The cosmetic and the dermatological or pharmaceutical preparations are generally based on a carrier which comprises at least one oil phase. However, preparations merely based on water are also possible. Accordingly, oils, creams, pastes, foams, preparations in stick form or grease-free gels or preferably emulsions are suitable.

Suitable emulsions are O/W emulsions, W/O emulsions, microemulsions or multiple emulsions, such as O/W/O emulsions or W/O/W emulsions with one or more retinoids according to the invention present in dispersed form, where the emulsions are obtainable, for example, by phase inversion technology, as in DE-A-197 26 121.

Customary cosmetic auxiliaries which may be suitable as additives for the cosmetic or pharmaceutical preparations are e.g. coemulsifiers, fats and waxes, stabilizers, thickeners, biogenic active ingredients, film formers, fragrances, dyes, perlizing agents, preservatives, pigments, electrolytes (e.g. magnesium sulfate) and pH regulators. Suitable coemulsifiers are preferably known W/O and also O/W emulsifiers, such as, for example, polyglycerol esters, sorbitan esters or partially esterified glycerides. Typical examples of fats are glycerides; waxes to be mentioned are, inter alia, beeswax, paraffin wax or microcrystalline waxes, if appropriate in combination with hydrophilic waxes. The stabilizers used may be metal salts of fatty acids, such as e.g. magnesium stearate, aluminum stearate and/or zinc stearate. Suitable thickeners are, for example, crosslinked polyacrylic acids and derivatives thereof, polysaccharides, in particular xanthan gum, guar guar, agar agar, alginates and tyloses, carboxymethylcellulose and hydroxyethylcellulose, also fatty alcohols, monoglycerides and fatty acids, polycrylates, polyvinyl alcohol and polyvinylpyrrolidone. Biogenic active ingredients are understood as meaning, for example, plant extracts, protein hydrolysates and vitamin complexes. Customary film formers are, for example, hydrocolloids, such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds. Suitable preservatives are, for example, formaldehyde solution, p-hydroxybenzoate or sorbic acid. Suitable pearlizing agents are, for example, glycol distearic esters, such as ethylene glycol distearate, but also fatty acids and fatty acid monoglycol esters. Dyes which can be used are the substances approved and suitable for cosmetic purposes, as listed, for example, in the publication “Kosmetische Färbemittel” [Cosmetic Colorants] from the Farbstoffkommission der Deutschen Forschungsgemeinschaft [Dyes Commission of the German Research Society], published by Verlag Chemie, Weinheim, 1984. These dyes are customarily used in a concentration of from 0.001 to 0.1% by weight, based on the total mixture.

The use of further antioxidants is advantageous in many cases. Thus, in addition to the antioxidants according to the invention specified at the start, it is possible to use all antioxidants which are customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thiorodoxin, glutathione, cystine, cystamine and glycosyl, N-acetyl, methyl, ethyl, propyl, amyl, butyl, and lauryl, palmitoyl, oleyl-, γ-linoleyl, cholesteryl and glyceryl esters) thereof) and also salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to ∝mol/kg), also (metal) chelating agents (e.g. α-hydroxy fatty acids, palmitic acid, phytic acid, lactoferrin), α-hydroxy acid (e.g. citric acid, lacetic acid, maleic acid), humic acid, bile acid, bile extracts, biliburin, biliverdin, EDTA and derivatives thereof, unsaturated fatty acids and derivatives thereof (e.g. γ-linolenic acid, linolic acid, oleic acid), folic acid and derivatives thereof, and coniferyl benzoate of benzoin resin, rutinic acid and derivatives thereof, α-glycosylrutin, ferulic acid, furfurylideneglucitol, carnosine, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The total amount of the abovementioned antioxidants (one or more compounds) in the preparations is preferably 0.075 to 30% by weight, particularly preferably 0.1 to 20% by weight, in particular 0.55 to 10% by weight, based on the total weight of the preparation.

Customary oil components in cosmetics are, for example, paraffin oil, glyceryl stearate, isopropyl myristate, diisopropyl adipate, cetylstearyl 2-ethylhexanoate, hydrogenated polyisobutene, Vaseline, caprylic/capric triglycerides, microcrystalline wax, lanolin and stearic acid.

Production Method:

There are numerous ways of producing a cosmetic preparation. For example, the hot/hot method, the hot/cold method or the cold/cold method, as described, for example, in “Kosmetik—Entwicklung, Herstellung und Anwendung kosmetischer Mittel” [Cosmetics—development, production and use of cosmetic compositions], ed. Wilfried Umbach, Thieme Verlag, 1995, page 511, are used. With the help of these methods it is possible to prepare oil-in-water (O/W), water-in-oil (W/O), but also multiple emulsions and cream gels and gels. The active ingredients are incorporated here preferably after the formulation has been cooled to below 40° C., in particularly sensitive cases, preferably after it has been cooled to room temperature. For the investigations which form the basis of this invention, an O/W emulsion was prepared in the hot/hot method and the active ingredients were then incorporated into the finished formulation at room temperature.

The Formulation Used:

| | % | Ingredient | INCI |
|---|---|---|---|
| Phase A | 2.00 | Cremophor A 6 | Ceteareth-6, Stearyl |
| | | | Alcohol |
| | 2.00 | Cremophor A 25 | Ceteareth-25 |
| | 3.00 | Jojoba oil | Simmondsia Chinensis |
| | | | (Jojoba) Seed Oil |
| | 3.00 | Cetylstearyl alcohol | Cetearyl Alcohol |
| | 10.00 | Paraffin oil, viscous | Mineral Oil |
| | 5.00 | Vaseline | Petrolatum |
| | 4.00 | Miglyol 812 | Caprylic/Capric Tri- |
| | | | glyceride |
| | 0.10 | BHT | BHT |
| Phase B | 5.00 | 1,2-Tropylene glycol | Propylene Glycol |
| | | USP | |
| | 0.10 | Edeta BD | Disodium EDTA |
| | 20.00 | Carbopol 934, | Carbomer |
| | | 1% in water dem. | |
| | 0.30 | Chemag 2000 | |
| | ad 100 | Water dem. | Aqua dem. |
| Phase C | 0.80 | Sodium hydroxide, | Sodium Hydroxide |
| | | 10% in water dem. | |
| Phase D | 0.50 | Vitamin E acetate | Tocopheryl Acetate |
| | 0.20 | Phenoxyethanol | Phenoxyethanol |
| | q.s. | Perfume oil | |

Production:

Phases A and B were heated separately to about 80° C. Phase B was then stirred into phase A and homogenized. The mixture was neutralized with phase C and after-homogenized. With stirring, the cream was cooled to about 40° C., phase D was stirred in and the mixture was homogenized again.

The water-soluble and oil-soluble antioxidants and also the UV filters were then, after cooling the cream to room temperature, incorporated into the finished emulsion. For this, firstly D-L-alpha-tocopherol and the UV filter were added, then the ascorbic acid or sodium ascorbate and subsequently retinol (Retinol 15D®, BASF; 15% strength solution of retinol in a medium-chain triglyceride) were incorporated with stirring.

The cream was then bottled either in aluminum tubes with an internal protective coating, or in light-permeable glass vessels.

In accordance with the production method described above, creams with varying amounts of retinol, (all-rac)-α-tocopherol and L-ascorbic acid and at least one UV filter were produced and stored for 12 weeks at 40° C. for stability investigations.

The subject matter of the invention is illustrated in more detail by reference to the following formulation examples.

### EXAMPLE 1

Skin lotion (O/W emulsion)

| | % by wt. |
|---|---|
| Ceteareth-6 and stearyl alcohol | 2.50 |
| Ceteareth-25 | 2.50 |
| Hydrogenated cocoglyceride | 1.50 |
| PEG-40 dodecyl glycol copolymer | 3.00 |
| Dimethicone | 3.00 |
| Phenethyl dimethicone | 2.00 |
| Cyclomethicone | 1.00 |
| Cetearyl octanoate | 5.00 |
| Avocado oil | 1.00 |
| Almond oil | 2.00 |
| Wheat germ oil | 0.80 |
| Panthenol USP | 1.00 |
| Phytantriol | 0.20 |
| Vitamin E acetate | 0.30 |
| Propylene glycol | 5.00 |
| Perfume | q.s. |
| Preservative | q.s. |
| Sodium ascorbate | 0.20 |
| Retinol 15D ® | 0.20 |
| Tocopherol | 0.10 |
| Diethylamino hydroxybenzoyl | 0.06 |
| hexyl benzoate (Uvinul ® A Plus) | |
| Water | ad 100 |

### EXAMPLE 2

Hand cream (W/O emulsion)

| | % by wt. |
|---|---|
| Cetearyl alcohol | 1.00 |
| Glyceryl stearate | 1.50 |
| Stearyl alcohol | 1.50 |
| Cetyl palmitate | 2.00 |
| Vitamin E acetate | 0.50 |
| Dimethicone | 8.00 |
| Ceteareth-6 and stearyl alcohol | 3.00 |
| Octyl methoxycinnamate | 5.00 |
| Propylene glycol | 8.00 |
| Panthenol | 1.00 |
| Evening primrose oil | 3.00 |
| PEG-7 hydrogenated castor oil | 6.00 |
| Glyceryl oleate | 1.00 |
| Phenethyl dimethicone | 3.00 |
| Beeswax | 1.50 |
| Carob seed grain | 0.80 |
| Silk powder | 0.80 |
| Preservative | q.s. |
| Perfume | q.s. |
| Borax | 0.10 |
| Sodium ascorbate | 0.30 |
| Tocopherol | 0.60 |
| Retinol 15D ® | 0.66 |
| Benzophenone-2 (Uvinul ® D 50) | 0.05 |
| Water | ad 100 |

### EXAMPLE 3

Sunscreen lotion (W/O emulsion)

| | % by wt. |
|---|---|
| PEG-7 hydrogenated castor oil | 6.00 |
| PEG-40 hydrogenated castor oil | 0.50 |
| Isopropyl palmitate | 7.00 |
| PEG-45/dodecyl glycol copolymer | 2.00 |
| Jojoba oil | 3.00 |
| Magnesium stearate | 0.60 |
| Octyl methoxycinnamate | 5.00 |
| C 12-15 alkyl benzoate | 5.00 |
| Titanium dioxide | 4.00 |
| Propylene glycol | 5.00 |
| EDTA | 0.20 |
| Preservative | q.s. |
| Retinol 15D ® | 0.33 |
| Water | ad 100 |
| Sodium ascorbyl phosphate | 1.00 |
| Vitamin E acetate | 0.50 |
| Sodium ascorbate | 0.20 |
| Tocopherol | 1.00 |
| Perfume | q.s. |

### EXAMPLE 4

Multiple Emulsion (W/O/W emulsion)

| | % by wt. |
|---|---|
| Paraffin oil | 7.50 |
| Cetearyl octanoate | 2.50 |
| Aluminum stearate | 0.25 |
| Magnesium stearate | 0.25 |
| Microcrystalline wax H | 0.50 |
| Cetearyl alcohol | 1.00 |
| Lanolin alcohol | 1.50 |
| Wool wax alcohol ointment | 1.50 |
| PEG-7 hydrogenated castor oil | 0.75 |
| PEG-45/dodecyl glycol copolymer | 2.00 |
| Ceteareth-6 and stearyl alcohol | 2.00 |
| Ceteareth-25 | 2.00 |
| Trilauret-4 phosphate | 1.00 |
| Hydroxyethylcellulose 0.20 | |
| Propylene glycol | 7.50 |
| Magnesium sulfate | 0.25 |
| Sodium ascorbate | 0.30 |
| Tocopherol | 0.01 |
| Retinol 15D ® | 0.40 |
| Diethylamino hydroxybenzoyl | 0.06 |
| hexyl benzoate (Uvinul ® A Plus) | |
| Water | ad 100 |

### EXAMPLE 5

Microemulsion

| | % by wt. |
|---|---|
| Ceteareth-25 | 13.00 |
| PEG-7 Glyceryl cocoate | 20.00 |
| Octyldodecanol | 5.00 |
| Preservative | q.s. |
| Sodium ascorbate | 0.10 |
| Tocopherol | 0.10 |
| Retinol 15D ® | 0.66 |
| Benzophenone-3 (Uvinul ® M 40) | 0.07 |
| Water | ad 100 |

### EXAMPLE 6

Liposom gel (hydrophilic gel)

| | % by wt. |
|---|---|
| PEG-40 hydrogenated castor oil | 1.00 |
| Bisabolol rac. | 0.10 |
| Propylene glycol | 8.00 |
| Panthenol | 0.50 |
| Water, vitamin E acetate, | 3.00 |
| Polysorbate 80 and caprylic/capric | |
| triglyceride and lecithin | |
| Preservative | q.s. |
| Perfume | q.s. |
| Carbomer | 0.50 |
| Sodium ascorbate | 0.15 |
| Tocopherol | 0.15 |
| Triethanolamine | 0.70 |
| Retinol 15D ® | 0.33 |
| Diethylamino hydroxybenzoyl | 0.06 |
| hexyl benzoate (Uvinul ® A Plus) | |
| Water | ad 100 |

### EXAMPLE 7

Make-up (decorative cosmetics)

| | % by wt. |
|---|---|
| Ceteareth-6 and stearyl alcohol | 9.00 |
| Dimethicone | 5.00 |
| Cetearyl octanoate | 8.00 |
| Makadamia nut oil | 5.00 |
| Propylene glycol | 5.00 |
| Retinol 15D ® | 0.66 |
| Water | ad 100 |
| Sicovit White E 171 | 8.00 |
| Sicomet Brown 70 13E 3717 | 2.00 |
| Sodium ascorbate | 0.20 |
| Tocopherol | 0.50 |
| Perfume | q.s. |
| Benzophenone-3 (Uvinul ® M 40) | 5.00 |

### EXAMPLE 8

Fluid make-up (decorative cosmetics)

| | % by wt. |
|---|---|
| Ceteareth-6 and stearyl alcohol | 7.00 |
| Ceteareth-25 | 5.00 |
| Dimethicone | 5.00 |
| Cetearyl octanoate | 8.00 |
| Makadamia nut oil | 5.00 |
| Propylene glycol | 5.00 |
| Retinol 15D ® | 0.33 |
| Water | ad 100 |
| Sicovit White E 171 | 8.00 |
| Sicomet Brown 70 13E 3717 | 1.00 |
| Sodium ascorbate | 0.10 |
| Tocopherol | 0.01 |
| Perfume | q.s. |
| Benzophenone-3 (Uvinul ® M 40) | 5.00 |

### EXAMPLE 9

Sunscreen lotion (O/W emulsion)

| | % by wt. |
|---|---|
| Ceteareth-6 and stearyl alcohol | 2.30 |
| Ceteareth-25 | 1.00 |
| Cetylstearyl alcohol | 4.80 |
| Cetylstearyl octanoate | 10.0 |
| Glyceryl monostearate | 3.00 |
| Vaseline | 3.00 |
| Panthenol USP | 1.00 |
| Disodium EDTA | 0.20 |
| Imidazolidinylurea | 0.30 |
| Propylene glycol | 5.00 |
| Sodium ascorbyl 2-monophosphate | 0.20 |
| Vitamin E acetate | 1.00 |
| Bisabolol | 0.10 |
| Retinol | 0.05 |
| Tocopherol | 0.20 |
| Sodium ascorbate | 0.20 |
| Diethylamino hydroxybenzoyl | 2.00 |
| hexyl benzoate (Uvinul ® A Plus) | |
| Ethylhexyl p-methoxycinnamate | 3.00 |
| Uvinul ® MC 80) | |
| Water | ad 100 |

1. A preparation comprising
a. at least one retinoid,
b. at least one water-soluble antioxidant,
c. at least one oil-soluble antioxidant and
d. 0.01 to 10% by weight of at least one UV filter,
wherein, in the preparation, per part by weight of retinoid, at least 1 part by weight of one or more water-soluble antioxidants and 0.1 to 100 parts by weight of one or more oil-soluble antioxidants are present, where the content of one or more water-soluble antioxidants is in the range from 0.05 to 0.8% by weight, based on the total amount of the preparation.

2. The preparation according to claim 1, which is a cosmetic or pharmaceutical preparation.

3. The preparation according to claims 1 and 2, comprising, per part by weight of retinoid, 1 to 6 parts by weight of one or more water-soluble antioxidants and 1 to 10 parts by weight of one or more oil-soluble antioxidants.

4. The preparation according to one of claims 1 to 3, comprising, per part by weight of retinoid, 3 to 5 parts by weight of one or more water-soluble antioxidants and 3 to 5 parts by weight of one or more oil-soluble antioxidants.

5. The preparation according to claims 1 to 4, comprising, as water-soluble antioxidant, L-ascorbic acid or salts of L-ascorbic acid.

6. The preparation according to claims 1 and 5, comprising α-tocopherol as oil-soluble antioxidant.

7. The preparation according to claims 1 to 8, wherein the retinoid is all-trans-retinol.

8. The preparation according to claim 7, comprising 0.015 to 0.2% by weight of all-trans-retinol.

9. The preparation according to claims 1 to 8, wherein the UV filters are UV-A, UV-B and/or broadband filters.

10. The preparation according to claims 1 to 9 in the form of an O/W, W/O or multiple emulsion.

11. The preparation according to claims 1 to 10, wherein it is stored in oxygen-impermeable packaging without the addition of protective gas.

12. The preparation according to claims 1 to 11, wherein it is a skincare preparation.
